(19) United States
(12) Patent Application Publication
(10) Pub. No.: US 2026/0334841 A1
(43) Pub. Date: Oct. 29, 2026

(54) ELECTRODE

(71) Applicant: Murata Manufacturing Co., Ltd., Nagaokakyo-shi (JP)

(72) Inventor: Kosuke SUGIURA, Nagaokakyo-shi (JP)

(21) Appl. No.: 19/699,219

(22) Filed: Jun. 5, 2026

Related U.S. Application Data
(63) Continuation of application No. PCT/JP2024/043608, filed on Dec. 10, 2024.

(30) Foreign Application Priority Data
Dec. 11, 2023 (JP) 2023-208790

(51) Int. Cl. A61B 5/268; A61B 5/291; G01N 27/327

(52) U.S. Cl. CPC A61B 5/268; A61B 5/291; G01N 27/327

(57) ABSTRACT

An electrode in which at least a contact surface with an object to be measured is formed of a conductive composite material comprising: a layered material particle comprising one or more layers, and an elastomer material. The one or more layers comprising a layer body represented by the following formula: MₘXₙ, wherein M is one or more metals selected from Group 3, 4, 5, 6, or 7 and includes Ti; X is a carbon atom, a nitrogen atom, or a combination thereof; n is not less than 1 and not more than 4; and m is greater than n but not more than. A modification or termination T is present on a surface of the layer body, and T is at least one selected from the group consisting of a hydroxyl group, a fluorine atom, a chlorine atom, an oxygen atom, and a hydrogen atom.

### CROSS REFERENCE TO RELATED APPLICATIONS

The present application is a continuation of International application No. PCT/JP2024/043608, filed Dec. 10, 2024, which claims priority to Japanese Patent Application No. 2023-208790, filed Dec. 11, 2023, the entire contents of each of which are incorporated herein by reference.

### TECHNICAL FIELD

The present disclosure relates to an electrode.

### BACKGROUND ART

Examples of an electrode for detecting a bioelectric signal such as an electroencephalogram signal from a subject include a soft electrode made of an elastomer material. For example, Patent Document 1 discloses a soft electrode (1) for measuring a bioelectric signal of a subject, the soft electrode (1) including a support (2) having a contact side (21) facing the subject when the electrode (1) is applied to the subject, and a connector side (22) opposite to the contact side (21).
Patent Document 1: WO 2022/047595

### SUMMARY OF THE DISCLOSURE

However, Patent Document 1 does not particularly consider an electrode having a further reduced impedance. An object of the present disclosure is to provide an electrode with further reduced impedance.

According to one aspect of the present disclosure, there is provided an electrode in which at least a contact surface with an object to be measured is formed of a conductive composite material containing a layered material particle including one or more layers, and an elastomer material.

The one or more layers including a layer body represented by the following formula: MₘXₙ, wherein M is one or more metals selected from Group 3, 4, 5, 6, or 7 and includes Ti; X is a carbon atom, a nitrogen atom, or a combination thereof, n is not less than 1 and not more than 4; and m is greater than n but not more than 5.

A modification or termination T is present on a surface of the layer body, and T is at least one selected from the group consisting of a hydroxyl group, a fluorine atom, a chlorine atom, an oxygen atom, and a hydrogen atom.

According to the present disclosure, it is possible to provide an electrode with further reduced impedance.

### BRIEF DESCRIPTION OF THE DRAWINGS

FIG. 1(a) and FIG. 1(b) are schematic sectional views showing layered material particles that can be used in one embodiment of the present disclosure, in which FIG. 1 (a) shows a single-layer MXene particle, and FIG. 1 (b) shows a multilayer (illustratively, two-layer) MXene particle.

FIG. 2 is a schematic sectional view showing one embodiment of the electrode of the present disclosure.

FIG. 3 is a schematic sectional view showing another embodiment of the electrode of the present disclosure.

FIG. 4 is a schematic sectional view showing another embodiment of the electrode of the present disclosure.

FIG. 5 is a schematic sectional view showing another embodiment of the electrode of the present disclosure.

FIG. 6 is a schematic sectional view showing another embodiment of the electrode of the present disclosure.

FIG. 7 is a schematic sectional view showing another embodiment of the electrode of the present disclosure.

FIG. 8 is a schematic sectional view showing another embodiment of the electrode of the present disclosure.

FIG. 9 is a schematic sectional view showing another embodiment of the electrode of the present disclosure.

FIG. 10 is a schematic sectional view showing another embodiment of the electrode of the present disclosure.

FIG. 11 is a schematic sectional view showing another embodiment of the electrode of the present disclosure.

### DETAILED DESCRIPTION

Hereinafter, an electrode in one embodiment of the present disclosure will be described in detail in a non-limiting manner.

### [Electrode]

The electrode of the present embodiment is an electrode in which at least a contact surface with an object to be measured is formed of a conductive composite material containing a layered material particle including one or more layers, and an elastomer material,
the one or more layers including a layer body represented by the following formula:

MₘXₙ
(wherein M is one or more metals selected from Group 3, 4, 5, 6, or 7 and includes Ti;
X is a carbon atom, a nitrogen atom, or a combination thereof,
n is not less than 1 and not more than 4; and
m is greater than n but not more than 5.)
and a modification or termination T (T is at least one selected from the group consisting of a hydroxyl group, a fluorine atom, a chlorine atom, an oxygen atom, and a hydrogen atom.) present on a surface of the layer body. As a result, it is possible to realize an electrode exhibiting low impedance containing a particle of a layered material (MXene) including one or more layers, and containing also an elastomer material. Since MXene exhibits higher conductivity than the carbon filler, even when MXene powder is dispersed in the elastomer material without adding carbon filler, for example, both low contact impedance required for an electrode such as a bioelectrode and impartation of conductivity to the elastomer material can be achieved.

### (Layered Material Particle)

Hereinafter, a layered material particle including one or more layers constituting the electrode of the present embodiment will be described.

The layered material constituting the layered material particle may be understood as a layered compound, is also represented by “MₘXₙTₛ” wherein s is any number, and x or z may be conventionally used instead of s. Typically, n can be 1, 2, 3, or 4, but is not limited thereto.

In the above formula of MXene, M may be only Ti, or may include Ti, and may further include one or more metals of Group 3, 4, 5, 6, or 7 other than Ti. For example, M may include Ti, and may further include at least one selected from the group consisting of Zr, Hf, V, Nb, Ta, Cr, Mo, and Mn. When M includes an element other than Ti, the element other than Ti is more preferably at least one selected from the group consisting of V, Cr, and Mo.

As MXene, those having the above formula MₘXₙ expressed as below are known.

Ti₂C, Ti₂N, (Ti,V)₂C, (Ti,Nb)₂C, Ti₃C₂, Ti₃N₂, Ti₃(CN), (Ti,V)₃C₂, (Ti₂Nb)C₂, (Ti₂Ta)C₂, (Ti₂Mn)C₂, (V₂Ti)C₂, (Cr₂Ti)C₂, (Mo₂Ti)C₂, (W₂Ti)C₂, Ti₄N₃, (Ti,Nb)₄C₃, (Ti₂Nb₂)C₃, (Ti₂Ta₂)C₃, (V₂Ti₂)C₃, (Cr₂Ti₂)C₃, (Mo₂Ti₂)C₃, (W₂Ti₂)C₃

Typically in the above formula, M can be titanium, or titanium and vanadium, and X can be a carbon atom or a nitrogen atom. For example, the MAX phase which is a precursor of MXene is Ti₃AlC₂ and MXene is Ti₃C₂Tₛ (in other words, M is Ti, X is C, n is 2, and m is 3).

In the present embodiment, MXene may include remaining A atoms in a relatively small amount, for example, at 10 mass % or less with respect to the original A atoms. The remaining amount of A atoms can be preferably 8 mass % or less, and more preferably 6 mass % or less. However, even if the remaining amount of A atoms exceeds 10 mass %, there may be no problem depending on the use and conditions of use of the electrode.

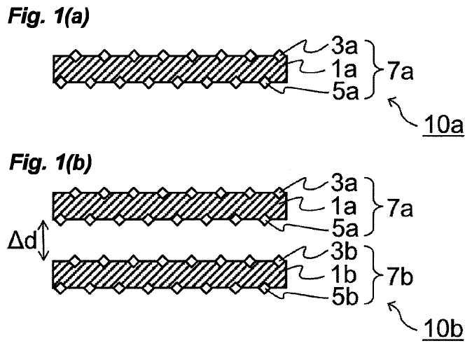

Hereinafter, a layered material particle (also referred to as “MXene particle”) included in the electrode according to the present embodiment will be described with reference to FIG. 1.

The layered material particle of the present embodiment is an aggregate containing one layer of a MXene 10a (single-layer MXene) schematically exemplified in FIG. 1(a). More specifically, the MXene 10a is a MXene layer 7a having a layer body (MₘXₙ layer) 1a represented by MₘXₙ and a modification or a termination T 3a, 5a present on a surface of the layer body 1a (more specifically, at least one of both surfaces, facing each other, of each layer). Therefore, the MXene layer 7a is also represented by “MₘXₙTₛ” wherein s is any number.

The layered material particle according to the present embodiment may include a plurality of layers together with one layer. As schematically illustrated in FIG. 1(b), examples of the MXene having plurality of layers (multilayer MXene) include, but are not limited to, a MXene 10b of two layers. In FIG. 1(b), 1b, 3b, 5b, and 7b are the same as 1a, 3a, 5a, and 7a in FIG. 1(a) described above. In the multilayer MXene, two adjacent MXene layers (e.g., 7a and 7b) may not necessarily be completely separated from each other, but may be partially in contact with each other. The MXene 10a exists as a single layer as a result of individual separation of the multilayer MXene 10b, and unseparated multilayer MXene 10b may remain, and the MXene 10a may be a mixture of the single-layer MXene 10a and the multilayer MXene 10b. From the viewpoint of ensuring the strength by containing the elastomer material while developing low impedance by MXene, it is preferable to contain multilayer MXene as the layered material particle according to the present embodiment.

Although the present embodiment is not limited, the thickness of each layer (corresponding to the above MXene layers 7a, 7b) of MXene can be, for example, not less than 1 nm and not more than 30 m, and may be, for example, not less than 1 nm and not more than 5 nm, and may further be not less than 1 nm and not more than 3 nm (these can differ depending on the number of M atomic layers included in each layer). For individual laminates of the multilayer MXene that may be included, the interlayer distance (or gap dimension, denoted as Δd in FIG. 1(b)) may be, for example, not less than 0.8 nm and not more than 10 nm, particularly not less than 0.8 nm and not more than 5 nm, and more particularly about 1 nm, and the total number of layers may be not less than 2 and not more than 20,000.

### (Elastomer Material)

The electrode of the present embodiment includes an elastomer material. MXene alone cannot be formed into, for example, the shape of an electrode such as a bioelectrode, and by containing an elastomer material together with MXene, an electrode shape can be easily formed. MXene exhibits a lower AC impedance than conventional materials. The electrode of the present embodiment includes MXene, and preferably includes multilayer MXene. In the multilayer MXene, MXene flakes are firmly bonded to each other as compared with single-layer MXene, so that the elastomer material is not inserted between layers. Since at least the surface between MXene flakes of the laminated MXene flakes in the multilayer MXene is not covered with the elastomer material, it is considered that the impedance can be reduced by using the multilayer MXene as MXene as compared with the case where the entire surface of single-layer MXene is covered with the elastomer material.

Examples of the elastomer material included in the electrode of the present embodiment include synthetic or natural rubbers, for example, butyl rubber, isoprene rubber, butadiene rubber, a halogenated butyl rubber (for example, bromobutyl rubber), an ethylene propylene terpolymer, silicone rubber, fluoro or perfluoro elastomer material, chlorosulfonic acid, polybutadiene, butyl rubber, neoprene, nitrile, polyisoprene, buna N, copolymer rubbers such as ethylene propylene (EPR), an ethylene-propylene-diene monomer (EPDM), acrylonitrile-butadiene (NBR or HNBR), and styrene-butadiene (SBR), a blend of ethylene or propylene-EPDM, EPR, or NBR, or a combination thereof. The term “synthetic rubber” also alternatively encompasses thermoplastic or thermosetting elastomer materials, such as polyurethane, silicone, fluorosilicone, styrene-isoprene-styrene (SIS), and styrene-butadiene-styrene (SBS), as well as materials that may be broadly classified as other polymers exhibiting rubber-like properties such as plasticized nylon, polyolefin, polyester, ethylene vinyl acetate, fluoropolymers, and polyvinyl chloride.

The volume ratio of the layered material particle to the total of the layered material particle and the elastomer material is preferably not less than 20 vol % and not more than 80 vol %. By setting the volume ratio of the layered material particle to 20 vol % or more, it becomes easy to reduce the impedance. The volume ratio is more preferably 25 vol % or more, further preferably 30 vol % or more. The higher the volume ratio of the layered material particle, the easier the impedance can be reduced. From the viewpoint of ensuring the strength of the electrode easily, it is preferable to set the volume ratio of the layered material particle to 80 vol % or less. The electrode of the present embodiment is made of the conductive composite material, and the volume ratio of the layered material particle in the conductive composite material is preferably not less than 20 vol % and not more than 80 vol %.

### (Metal Atom)

The layered material particle of the present embodiment is preferably a metal atom-containing layered material particle containing a metal atom. By containing a metal atom, the electric double layer capacitance is further increased, and the impedance can be further lowered. The metal atom is preferably an alkali metal atom whose cation is monovalent rather than a metal atom whose cation is divalent. The metal atom is preferably one or more of lithium, sodium, and potassium. The metal atom is preferably present between layers of MXene by intercalation of the metal atom. The metal atom may be in the state of a metal cation in MXene. That is, the metal cation can exist between layers of MXene as a monovalent metal cation.

It is preferable that the metal atom is contained in an amount of preferably 6.0×10⁻⁴ mol or more per 1 g of the metal atom-containing layered material particle, because the impedance can be further reduced while the adhesion strength with a base material or the like is maintained.

### (Modes of Electrode)

The electrode of the present embodiment may be an electrode in which at least the contact surface with an object to be measured is formed of a conductive composite material containing a predetermined layered material particle and an elastomer material. Hereinafter, some specific modes of the electrode of the present embodiment will be exemplified in a non-limiting manner.

One of preferable modes of the electrode of the present embodiment is a brush-shaped electrode including a support and a plurality of contact pins to be brought into contact with an object to be measured, in which the contact surface with an object to be measured is at least a part of a surface of at least one contact pin among the plurality of contact pins.

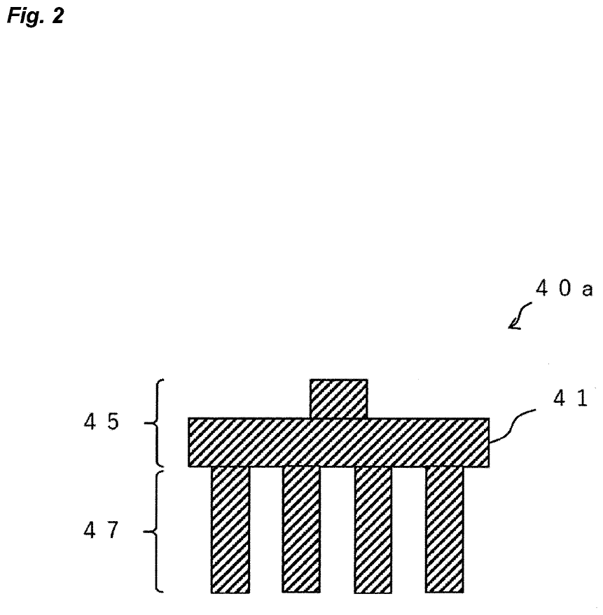

An example of the electrode of the present embodiment is an electrode made of a conductive composite material. For example, as shown in the schematic sectional view of FIG. 2, a brush-shaped electrode 40a having a structure in which MXene is dispersed throughout the elastomer material can be mentioned. The volume ratio of the layered material particle in the conductive composite material is preferably not less than 20 vol % and not more than 80 vol %.

The electrode of the present embodiment may have a base material part containing carbon and an elastomer material. Examples of the base material part containing carbon and an elastomer material include a base material part substantially consisting of carbon and an elastomer material that contains carbon and an elastomer material, and other ingredient of, for example, 5 vol % or less, and further 1 vol % or less, and a base material part consisting of carbon and an elastomer material. The volume ratio of carbon in the base material part is preferably not less than 20 vol % and not more than 60 vol %, more preferably not less than 20 vol % and not more than 40 vol %, and further preferably not less than 20 vol % and not more than 35 vol %. The mass ratio of carbon in the base material part is preferably not less than 35 mass % and not more than 45 mass %.

The electrode of the present embodiment further has a base material part containing carbon and an elastomer material, the volume ratio of carbon in the base material part is preferably not less than 20 vol % and not more than 60 vol %, and the volume ratio of the layered material particle in the conductive composite material is preferably not less than 20 vol % and not more than 80 vol %.

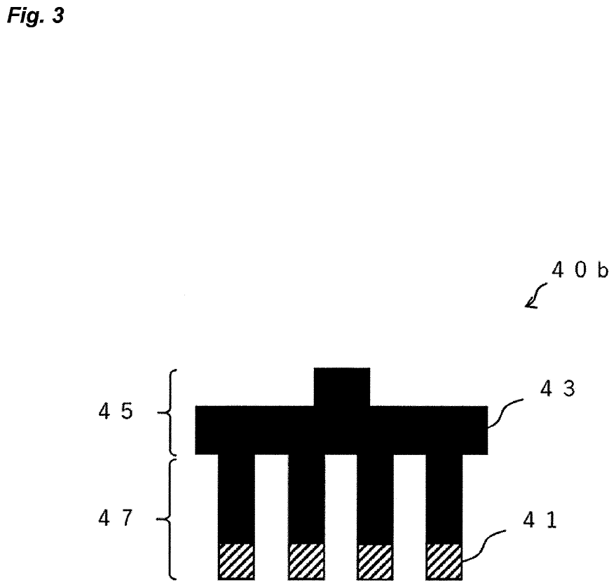

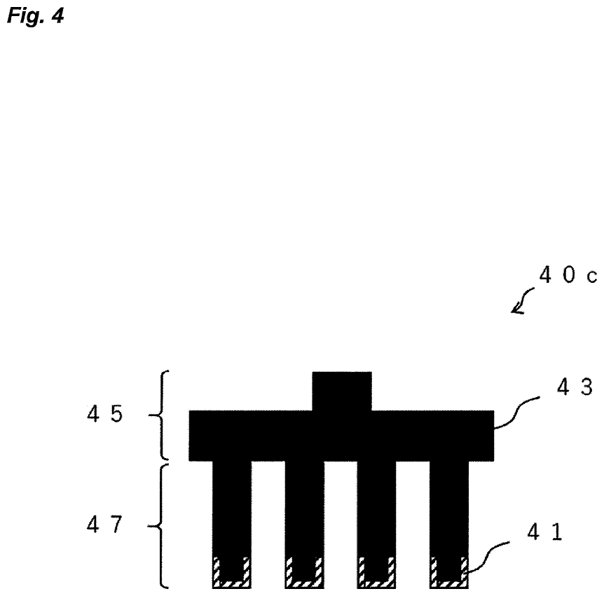

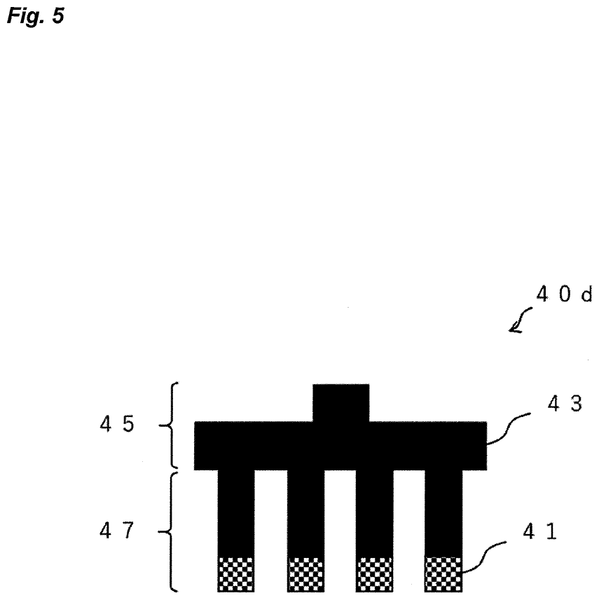

As other examples of the electrode of the present embodiment, examples of an electrode having a base material part made of carbon and an elastomer material are shown in FIGS. 3 to 5. FIGS. 3 to 5 are schematic sectional views of electrodes 40b, 40c, and 40d having a region 41 formed of a conductive composite material and a base material part 43 made of carbon and an elastomer material. For example, as shown in FIGS. 3 to 5, examples include brush-shaped electrodes having a support 45 and a plurality of contact pins 47 to be brought into contact with an object to be measured, in which as a region including a contact surface with an object to be measured, at least a part of the surface (the tip in FIGS. 3 and 5, and the surface of the tip in FIG. 4) of the contact pin 47 is the region 41 formed of a conductive composite material. In FIG. 5, the tip of the contact pin 47 is made of a conductive composite material 41 obtained by impregnating a porous material made of carbon and an elastomer material with MXene.

Although not illustrated, there is also a mode in which the tip of the contact pin 47 is porous in the electrode made of the conductive composite material in FIG. 2. In this mode, the porous tip of the contact pin may or may not be impregnated with MXene.

In FIGS. 3 to 5, tips of all the contact pins are formed of the conductive composite material, but the disclosure is not limited thereto, and at least one contact pin of the plurality of contact pins may have a region formed of a conductive composite material. In FIGS. 3 to 5, the support 45 and a region other than the region 41 formed of the conductive composite material in the contact pin 47 constitute the base material part made of carbon and an elastomer material in a non-limiting manner. For example, a conductive material other than carbon may be used instead of the carbon.

The conductive composite material preferably has a thickness of 1 μm or more in the depth direction from the contact surface with an object to be measured. For example, as shown in FIG. 4, a preferable mode is that the conductive composite material has a thickness of 1 μm or more in the depth direction from the surface of a part of the contact pin 47 which is a contact surface with an object to be measured. The thickness may be, for example, 10 μm or more, and further 50 μm or more, but if the thickness is too large, the surface of the electrode is easily peeled off, and thus the thickness is preferably 1 mm or less. It is preferable that the electrode of the present embodiment further has a base material part containing carbon and an elastomer material, the volume ratio of the carbon in the base material part is not less than 20 Vol % and not more than 60 Vol %, and the conductive composite material has a thickness of 1 μm or more in the depth direction from the contact surface with an object to be measured.

The volume ratio of the layered material particle in the conductive composite material is preferably not less than 20 Vol % and not more than 80 vol %. In the brush-shaped electrode as shown in FIGS. 3 to 5, the volume ratio of the carbon in the base material part is preferably not less than 20 vol % and not more than 60 vol %. Also, in the brush-shaped electrode as shown in FIGS. 3 to 5, the volume ratio of the layered material particle in the conductive composite material is preferably not less than 20 vol % and not more than 80 vol %.

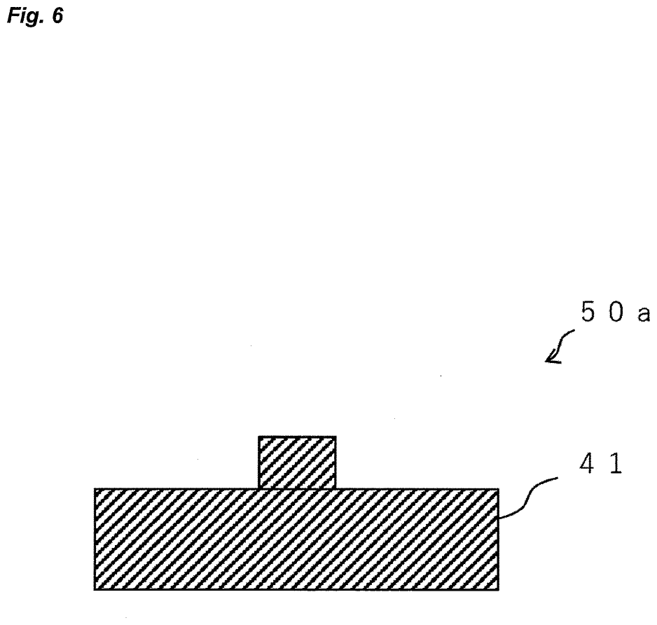

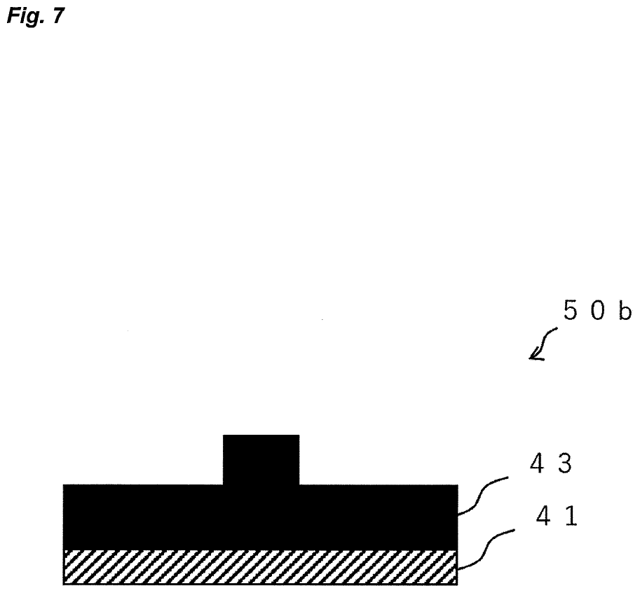

Another preferable mode of the electrode of the present embodiment is a disk-shaped electrode, in which the contact surface with an object to be measured is at least a part of a circular surface having a disk shape. FIGS. 6 and 7 illustrate schematic sectional views of a disk-shaped electrode. FIG. 6 shows a disk-shaped electrode which is an electrode 50a formed of the region 41 formed of a conductive composite material and shows a disk-shaped electrode having a structure in which MXene is dispersed in the entire elastomer material. FIG. 7 shows a disk-shaped electrode 50b having the region 41 formed of a conductive composite material, and the base material part 43 made of carbon and an elastomer material. In FIG. 7, a circular surface including a contact surface with an object to be measured is formed of a conductive composite material. In the disk-shaped electrode as shown in FIG. 7, the volume ratio of the carbon in the base material part is preferably not less than 20 vol % and not more than 60 vol %. Also, in the disk-shaped electrode as shown in FIG. 6 and FIG. 7, the volume ratio of the layered material particle in the conductive composite material is preferably not less than 20 vol % and not more than 80 vol %.

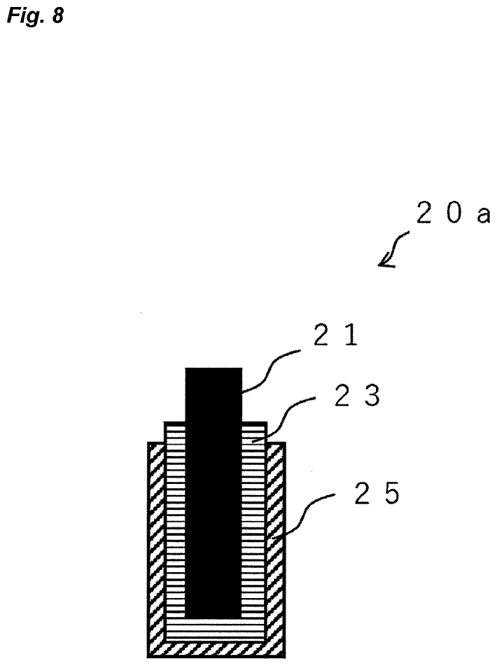

Still another preferable mode of the electrode of the present embodiment is an electrode further including a silver/silver chloride electrode part. FIGS. 8 to 11 illustrate schematic sectional views of an electrode further including a silver/silver chloride electrode part.

As one preferable mode, the electrode according to the present embodiment has a region formed of a conductive composite material, a base material part, and a silver/silver chloride electrode part. One mode of the electrode according to the present embodiment is an electrode in which a silver/silver chloride electrode part is disposed on at least a part of the surface of the base material part, and a region formed of a conductive composite material is disposed as a conductive film on at least a part of the surface of the silver/silver chloride electrode part exposed to the external environment. For example, as shown in a schematic sectional view in FIG. 8, an electrode 20a in which a silver/silver chloride electrode part 23 is disposed on the surface of an electrode base material 21, and a region (conductive film) 25 formed of a conductive composite material is disposed on the surface of the silver/silver chloride electrode part 23 is exemplified. As shown in FIG. 8, an electrode is exemplified in which the base material has a tip part and extends in a longitudinal direction, the silver/silver chloride electrode part 23 is disposed on the surfaces of the tip part and the side face part of the base material, and the region formed of the conductive composite material (conductive film) 25 is disposed on about 50% or more of the surface of the silver/silver chloride electrode part 23 exposed to the external environment.

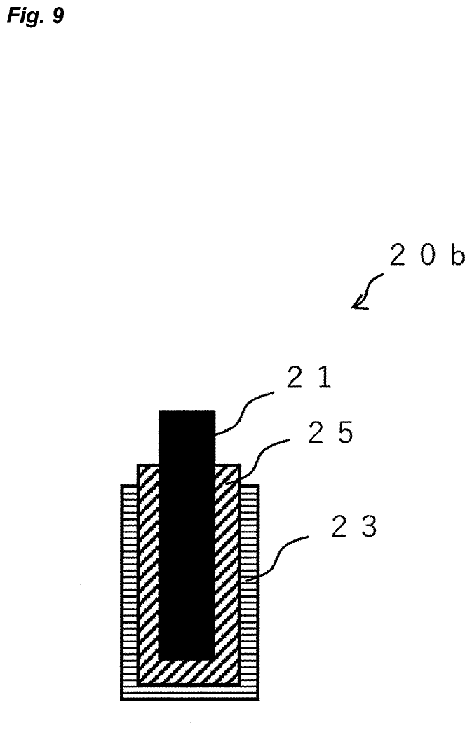

Another mode of the electrode according to the present embodiment is an electrode in which a region formed of a conductive composite material (conductive film) is disposed on at least a part of the surface of the base material part, and a silver/silver chloride electrode part is disposed on at least a part of the surface of the region formed of a conductive composite material (conductive film) exposed to the external environment. As an example thereof, for example, as shown in a schematic sectional view in FIG. 9, an electrode 20b in which a region formed of the conductive composite material (conductive film) 25 is disposed on the surface of the electrode base material 21, and the silver/silver chloride electrode part 23 is disposed on the surface of the region formed of a conductive composite material (conductive film) 25 is exemplified. As shown in FIG. 9, an electrode is exemplified in which the base material has a tip part and extends in a longitudinal direction, the region formed of the conductive composite material (conductive film) 25 is disposed on the surface including the tip part of the base material, and the silver/silver chloride electrode part 23 is disposed on about 50% or more of the surface of the region formed of the conductive composite material (conductive film) 25 exposed to the external environment.

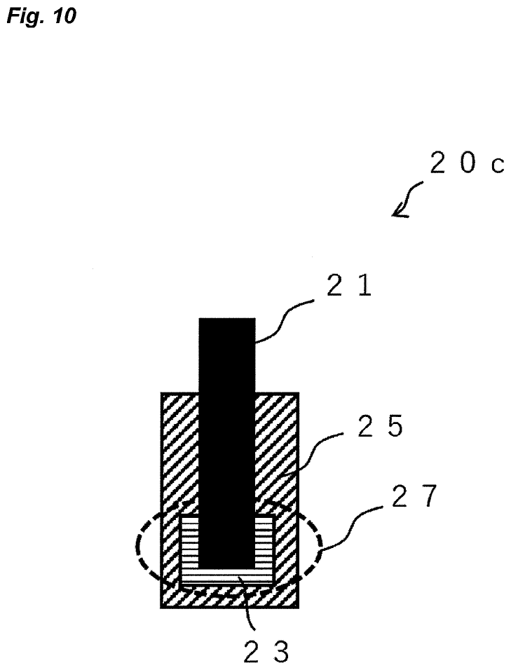

As a modified example of FIG. 8, as shown in the schematic sectional view of FIG. 10, an electrode 20c in which the ratio of the silver/silver chloride electrode part 23 disposed on the surface of the electrode base material 21 is smaller than that in FIG. 8 can be mentioned. As shown in FIG. 10, an electrode is exemplified in which the base material has a tip part and extends in a longitudinal direction, the silver/silver chloride electrode part 23 is disposed on the surface including the tip part of the base material, and the region formed of the conductive composite material (conductive film) 25 is disposed on the entire surface of the silver/silver chloride electrode part 23 exposed to the external environment. The electrode of the present embodiment is, for example, a contact pin in which the base material has a tip part and extends in a longitudinal direction as shown in FIG. 10, and the silver/silver chloride electrode part 23 is provided only in the tip region 27 of the contact pin, and the region formed of the conductive composite material (conductive film) 25 is disposed so as to cover the entire surface of the silver/silver chloride electrode part 23 exposed to the external environment. When the electrode of such a mode is used, for example, as a biosensing electrode, the electrode is excellent in biocompatibility because the surface to be in contact with the skin is the region formed of the conductive composite material (conductive film). In addition, as shown in FIG. 10, it is considered that by suppressing the ratio of the silver/silver chloride electrode part 23 in the electrode and covering the silver/silver chloride electrode part 23 with the region formed of the conductive composite material (conductive film) 25, deterioration of the silver/silver chloride electrode part 23 is suppressed, and the life of the electrode can be extended.

In the electrode according to the present embodiment, as shown in FIG. 8 and the like, the silver/silver chloride electrode part and the conductive film are preferably in direct contact at least partially.

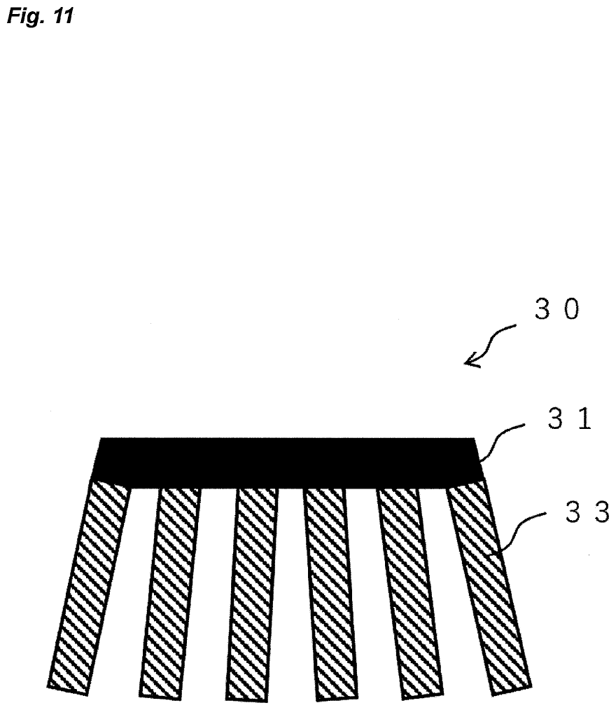

Another mode of the electrode according to the present embodiment is a brush-shaped electrode having a support and a plurality of contact pins to be brought into contact with an object to be measured, in which the silver/silver chloride electrode part is disposed on at least a part of the surface of the base material in a region including tips of the contact pins, and the region formed of a conductive composite material (conductive film) is disposed on at least a part of the surface of the silver/silver chloride electrode part exposed to the external environment. For example, as illustrated in FIG. 11, a brush-shaped electrode 30 in which a plurality of contact pins 33 are connected to a support 31 is exemplified, and each of the plurality of contact pins 33 can take any one of the modes of FIGS. 8 to 11 described above, for example.

FIGS. 2 to 11 are explanatory diagrams for easy understanding, and the shape, the size, the relative size ratios, the number of contact pins, intervals, the arrangement and the like of the region formed of a conductive composite material, the base material part, and the silver/silver chloride electrode part in these drawings may be different from those of actual electrodes. The electrode according to the present embodiment has been described with reference to the drawings, but the electrode according to the present embodiment is not limited to the modes illustrated in the drawings. For example, the cross section of the contact pin of the brush-shaped electrode may be not only the rectangular shape illustrated in the above drawings but also a tapered triangular shape or a tapered trapezoidal shape. In addition, the tip of the contact pin may be a flat surface or a convex curved surface.

For example, when an electrode is brought into contact with a part having hair as an electroencephalogram electrode in a dry state, it is preferable that the electrode has a brush shape as illustrated in FIG. 2 or the like, like a brush for brushing hair. On the other hand, a flat-shaped electrode as illustrated in FIG. 6 or the like is also suitable for a hairless part such as a forehead or a periphery of an ear. For example, the electrode of the present embodiment that can be used in such a manner that the electrode is in contact with the region includes an elastomer material that is a soft material so as not to cause pain in the scalp. Since the elastomer material itself has neither conductivity nor biological signal sensing function, as described above, it is possible to impart conductivity and biological signal sensing function by dispersing MXene. From the viewpoint of further enhancing the biological signal sensing sensitivity, it is preferable that the MXene includes a multilayer MXene having a multilayer structure. The inclusion of the multilayer MXene is preferable because a sensing function can be exhibited at the part not in contact with the elastomer material in the multilayer structure.

### [Manufacturing Method for Electrode]

The electrode according to the present embodiment can be manufactured, for example, by a method described below. However, the manufacturing method for an electrode according to the present embodiment is not limited to this embodiment.

A manufacturing method comprises:
(a) preparing a precursor represented by the following formula:

MₘAXₙ
(wherein M is one or more metals selected from Group 3, 4, 5, 6, or 7 and includes Ti;
X is a carbon atom, a nitrogen atom, or a combination thereof,
A is one or more elements selected from Group 12, 13, 14, 15, or 16;
n is not less than 1 and not more than 4; and
m is greater than n but not more than 5);
(b) performing an etching treatment of removing at least a part of A atoms from the precursor by using an etching solution containing F⁻;
(c) washing an etching treatment product obtained by the etching treatment with water to obtain a layered material particle; and
(d) forming a mixture of the layered material particle and an elastomer material to form at least part of an electrode. By this manufacturing method, an electrode with low impedance can be produced.

### Step (a)

First, a predetermined precursor is prepared. The predetermined precursor that can be used in the present embodiment is a MAX phase that is a precursor of MXene, and is represented by the following formula:

MₘAXₙ
(wherein M is one or more metals selected from Group 3, 4, 5, 6, or 7 and includes Ti;
X is a carbon atom, a nitrogen atom, or a combination thereof,
A is one or more elements selected from Group 12, 13, 14, 15, or 16;
n is not less than 1 and not more than 4; and
m is greater than n but not more than 5).

M, X, n, and m are as described above for MXene. A is one or more elements selected from Group 12, 13, 14, 15, or 16, and is usually an element of Group A, typically Group IIIA and Group IVA, and more particularly may contain at least one selected from the group consisting of Al, Ga, In, Tl, Si, Ge, Sn, Pb, P, As, S, and Cd, and is preferably Al.

The MAX phase has a crystal structure in which a layer formed by A atoms is located between two layers represented by MₘXₙ (which may have such a crystal lattice that each X is located in an octahedral array of M). When typically m=n+1, but not limited thereto, the MAX phase includes repeating units in which each one layer of X atoms is disposed in between adjacent layers of n+1 layers of M atoms (these are also collectively referred to as an “MₘXₙ layer”), and a layer of A atoms (“A atom layer”) is disposed as a layer next to the (n+1)th layer of M atoms.

The MAX phase can be produced by a known method. For example, a TiC powder, a Ti powder, and an Al powder are mixed with a ball mill, and the resulting mixed powder is fired in an Ar atmosphere to obtain a fired body (block-like MAX phase). Thereafter, the fired body obtained is crushed with an end mill to obtain a powdery MAX phase for the next step.

### Step (b)

An etching treatment for removing at least a part of A atoms from the precursor using an etching solution containing F⁻ is performed.

By selectively etching (removal and layer separation in some cases) A atoms (and part of M atoms) from the MAX phase, the A atom layer (and part of M atoms in some cases) is removed, and the surface of the exposed MₘXₙ layer is modified by hydroxyl groups, fluorine atoms, chlorine atoms, oxygen atoms, hydrogen atoms, etc., existing in an etching liquid (usually, an aqueous solution of a fluorine-containing acid is used, but not limited thereto), so that the surface is terminated.

The etching can be carried out using an etching liquid containing F⁻, and a method using, for example, a mixed liquid of lithium fluoride and hydrochloric acid, a method using hydrofluoric acid, or the like may be used.

In the case of obtaining a metal atom-containing layered material particle, the etching solution contains a metal compound containing a monovalent metal ion, and an intercalation treatment of the monovalent metal ion may be performed simultaneously with the etching. Examples of the metal compound containing a monovalent metal ion include those used in the following intercalation treatment. The content of the metal compound containing a monovalent metal ion in the etching solution is preferably 0.001 mass % or more. The content is more preferably 0.01 mass % or more, and further preferably 0.1 mass % or more. On the other hand, from the viewpoint of dispersibility in the solution, the content of the metal compound containing a monovalent metal ion in the etching solution is preferably 10 mass % or less, and more preferably 1 mass % or less.

### Step (c)

The etching treatment product obtained by the etching treatment is washed with water to obtain a layered material particle. After the etching, layer separation of MXene (delamination, separation of multilayer MXene into single-layer MXene) may be appropriately promoted by any suitable post-treatment (e.g., ultrasonic treatment, manual shaking, automatic shaker, or the like). In the case of obtaining multilayer MXene as the multilayer material particle, it is preferable not to perform delamination. Since the shear force of an ultrasonic treatment is so large that the MXene can be destroyed, it is desirable to apply an appropriate shear force by manual shaking, an automatic shaker or the like, when it is desired to obtain a two-dimensional MXene (preferably single-layer MXene) having a larger aspect ratio.

For the purpose of separating layers of MXene and obtaining a metal atom-containing layered material particle, the following intercalation treatment and delamination may be further performed.

### (Intercalation Treatment)

For example, an intercalation treatment of monovalent metal ion including a step of mixing the etching treatment product obtained by the etching treatment with a metal compound containing a monovalent metal ion may be performed. Examples of the monovalent metal ion constituting the metal compound containing a monovalent metal ion include alkali metal ions such as a lithium ion, a sodium ion, and a potassium ion, a copper ion, a silver ion, and a gold ion. Examples of the metal compound containing a monovalent metal ion include an ionic compound in which the metal ion and a cation are bonded. Examples include an iodide, a phosphate, a sulfur-containing salt such as a sulfate, a nitrate, an acetate, and a carboxylate of the metal ion. As described above, the monovalent metal ion is preferably a lithium ion, and the metal compound containing a monovalent metal ion is preferably a metal compound containing a lithium ion, more preferably an ionic compound of a lithium ion, and further preferably one or more of an iodide, a phosphate or a sulfide of a lithium ion. When a lithium ion is used as the metal ion, it is considered that water hydrating the lithium ion has the most negative dielectric constant, and thus it is easy to form a monolayer.

The content of the metal compound containing a monovalent metal ion in the formulation for intercalation treatment of monovalent metal ion is preferably 0.001 mass % or more. The content is more preferably 0.01 mass % or more, and further preferably 0.1 mass % or more. On the other hand, from the viewpoint of dispersibility in the solution, the content of the metal compound containing a monovalent metal ion is preferably 10 mass % or less, and more preferably 1 mass % or less.

### (Delamination)

Performing delamination using an intercalation treatment product obtained by intercalation can be mentioned. For example, delamination including the step of centrifuging the intercalation treatment product, discarding the supernatant, and then washing the remaining precipitate with water can be mentioned. Conditions for the delamination treatment are not particularly limited. The dispersion medium used for delamination is not particularly limited, and for example, delamination may be performed using one or more of a polar organic dispersion medium and an aqueous dispersion medium. The process of adding one or more of the polar organic dispersion medium and the aqueous dispersion medium, stirring, performing centrifugation, and recovering the supernatant liquid is repeated once or more times, preferably twice or more times but ten or less times to obtain a supernatant liquid containing a single-layer/few-layer MXene as a delamination treatment product. Alternatively, the supernatant may be centrifuged, and the supernatant after centrifugation may be discarded to obtain a clay containing single-layer/few-layer MXene as a delamination treatment product. In the case of obtaining multilayer MXene as the multilayer material particle, it is preferable not to perform delamination.

### (Powderization)

The slurry subjected to the steps of the intercalation treatment and the delamination treatment is freeze-dried to obtain a powder of MXene.

MXene exhibits a lower AC impedance than conventional materials. In order to maintain the conductivity of MXene, it is preferable to use the MXene dispersed in water slurry without drying the MXene. This is because when dried, MXene flakes aggregate and the arrangement is disturbed. However, since the contact impedance decreases as the area of contact between the MXene surface and the electrolytic solution such as human sweat increases, from the viewpoint of reducing the contact impedance, it is preferable to have a structure in which the arrangement of the MXene flakes is disturbed and the electrolytic solution can easily access it. Therefore, for example, in the application of an electrode such as a bioelectrode, MXene in a dry powder state may be used, and there is a case where it is better to use MXene in a dry powder state.

### Step (d)

A mixture of the layered material particle and the elastomer material is molded to form at least a part of the electrode. In order to mix the MXene particle and the elastomer material and mold the mixture into the shape of the electrode or the base material part, for example, an extruder is used, and the MXene and the elastomer material are introduced into a hopper and heated while the raw material is conveyed to melt, knead, and pressurize the mixture, and then for example, a brush-shaped electrode mold as shown in FIG. 2 or a disk-shaped electrode mold as shown in FIG. 6 is set, and extrusion molding is performed to obtain a brush-shaped electrode as shown in FIG. 2 or a disk-shaped electrode as shown in FIG. 6.

Alternatively, the following manufacturing method for an electrode can be mentioned. That is, in order to mix carbon and an elastomer material and mold the mixture into the shape of the electrode or the base material part, for example, an extruder is used, and the carbon and the elastomer material are introduced into a hopper and heated while the raw material is conveyed to melt, knead, and pressurize the mixture, and then for example, a brush-shaped electrode mold as shown in FIG. 2 or a disk-shaped electrode mold as shown in FIG. 6 is set, and extrusion molding is performed to first obtain a brush-shaped electrode as shown in FIG. 2 or a disk-shaped electrode as shown in FIG. 6. Thereafter, the tip of the contact pin of the brush-shaped electrode as shown in FIG. 2 and the circular surface of the disk-shaped electrode as shown in FIG. 6 are immersed in a mixture of the MXene particle and an elastomer material heated to, for example, 200 °C (for example, immersed for 5 to 30 minutes), whereby the surface of the tip of the contact pin of the electrode melts, and the electrode as shown in FIG. 4 or the electrode as shown in FIG. 7 can be obtained which includes a region formed of the conductive composite material formed of the MXene particle, carbon, and the elastomer material. By varying the time of the immersion, the thickness of the conductive composite material can be varied. By immersion for a long time, the conductive composite material is sufficiently immersed, and an electrode as shown in FIG. 3 can be obtained.

In order to make tips of contact pins porous as shown in FIG. 5, for example, after making a brush-shaped electrode made of carbon and an elastomer material, the tips of the contact pins are immersed in, for example, an inorganic acid to form voids and make the tips porous, followed by immersion in a MXene dispersion liquid at room temperature. After making the tip part of the electrode (for example, a soft electrode) porous in advance, the tip part is impregnated with a MXene slurry by capillary action, and dried to obtain an electrode in which a conductive composite material in which MXene is dispersed in the porous material and which exhibits lower contact impedance is formed on the contact surface with an object to be measured (for example, a living body).

A manufacturing method for an electrode having a silver/silver chloride electrode part of FIGS. 8 to 11 includes:

Forming a film containing a conductive composite material on at least a part of the surface (surface exposed to the external environment) of a silver/silver chloride electrode part formed on the surface of a base material part using the conductive composite material or forming a film containing the conductive composite material on at least a part of the surface of the base material part using the conductive composite material, and then forming a silver/silver chloride electrode part on at least a part of the surface (surface exposed to the external environment) of the film containing a particle of a two-dimensional material.

Stirring of the layered material particle and the elastomer material for obtaining the conductive composite material can be performed using a dispersing device such as a homogenizer, a propeller stirrer, a thin film swirling stirrer, a planetary mixer, a mechanical shaker, or a vortex mixer.

Drying after molding may be performed under mild conditions such as natural drying (typically, placing in an air atmosphere at normal temperature and normal pressure) or air drying (blowing air), or may be performed under relatively active conditions such as hot air drying (blowing heated air), heat drying, and/or vacuum drying. In the present embodiment, “drying” means removing the medium liquid that may be present in the precursor. The drying may be performed at a temperature of 400 °C or less using, for example, a normal pressure oven or a vacuum oven. Drying may be performed, for example, at not lower than 30 °C and not higher than 200 °C for not less than 30 minutes and not more than 24 hours.

Although the electrode of the present disclosure has been described in detail above, various modifications are possible. It should be noted that the electrode of the present disclosure may be produced by a method different from the manufacturing method in the above-described embodiment.

### EXAMPLES

Hereinafter, the present disclosure will be described more specifically with reference to examples. The present disclosure is not limited by the following examples, and can be implemented with appropriate modifications within a range that can be consistent with the above-described and later-described gist, and any of them is included in the technical scope of the present disclosure.

### Example 1

### 1. Preparation of MXene

First, a layered material particle (MXene particle) was obtained by sequentially performing (1) preparation of precursor (MAX), (2) etching of precursor, (3) cleaning after etching, (4) Li intercalation, (5) cleaning after intercalation treatment, and (6) powderization, which are described in detail below.

### (1) Preparation of Precursor (MAX)

TiC powder, Ti powder, and Al powder (all manufactured by Kojundo Chemical Laboratory Co., Ltd.) were placed in a ball mill containing zirconia balls at a molar ratio of 2:1:1 and mixed for 24 hours. The obtained mixed powder was fired in an Ar atmosphere at 1350 °C for 2 hours. The fired body (block-like MAX) thus obtained was crushed with an end mill to a maximum size of 40 μm or less. Thereby, a Ti₃AlC₂ particle was obtained as a precursor (powdery MAX).

### (2) Etching of Precursor (MAX)

Using the Ti₃AlC₂ particle (powder) prepared by the above method, etching was performed under the following etching conditions to obtain a solid-liquid mixture (slurry) containing a solid component derived from the Ti₃AlC₂ powder.

### (Etching Conditions)

Precursor: Ti₃AlC₂ (passed through a sieve having an opening size of 45 m)
Etching solution composition: 49% HF 6 mL,
H₂O 18 mL
HCl(12M) 36 mL
Amount of precursor input: 3.0 g
Etching container: 100 mL iboy
Etching temperature: 35 °C
Etching time: 24 h
Stirrer rotation speed: 400 rpm
   (3) Cleaning after Etching

The above slurry was equally divided into two portions and inserted into two 50 mL centrifuge tubes. Then, centrifugation was performed at 3500 G for 5 minutes using a centrifuge, and then the supernatant was discarded. Thereafter, (i) 35 mL of pure water was added to the remaining precipitate in each centrifuge tube, (ii) stirring was performed by manually shaking, (iii) centrifugation was performed at 3500 G for 5 minutes, and (iv) the supernatant was removed. The steps (i) to (iv) were repeated 10 times. Finally, centrifugation was performed at 3500 G for 5 minutes to obtain a Ti₃C₂Tₛ-water medium clay.

### (4) Li Intercalation

The Ti₃C₂Tₛ-water medium clay prepared by the above method was stirred at not lower than 20 °C and not higher than 25 °C for 12 hours using LiCl as a Li-containing compound according to the following conditions of Li intercalation to perform Li intercalation. The detailed conditions of Li intercalation are as follows.

### (Conditions of Li Intercalation)

Ti₃C₂Tₛ-water medium clay (cleaned MXene): solid content 0.75 g
LiCl: 0.75 g
Intercalation container: 100 mL iboy
Temperature: not lower than 20 °C and not higher than 25 °C (room temperature)
Time: 12 h
Stirrer rotation speed: 800 rpm
   (5) Cleaning after Intercalation Treatment

Each of the slurries obtained by the intercalation was transferred to a centrifuge tube, (i) 40 mL of pure water was added, (ii) centrifugation was performed under the condition of 3500 G using a centrifuge, and (iii) a supernatant was separated and removed. The operations (i) to (iii) were repeated five times in total to remove excessive Na, thereby obtaining a Li intercalated MXene clay as an intercalation treatment product.

### (6) Powderization

The intercalation treatment product was appropriately divided into petri dishes and frozen at −18 °C or lower. Thereafter, the intercalation treatment product was dried with a freeze dryer for 24 hours or more to obtain a powder (MXene powder).

2-1. Preparation of Electrode in which MXene is Entirely Dispersed

The MXene powder obtained in the above (6) and chips of an elastomer material EPDM were weighed so that the content of the MXene (MXene volume ratio) in the mixture was a value shown in Tables 1 and 2, and were mixed in a dry manner to obtain a mixture. The mixture was charged into an injection molding machine, and an elastomer material in a state in which MXene powder was dispersed was injected into a metal mold in which a shape of a rubber base material was carved. After the injection, a molded product obtained by extrusion from the metal mold was obtained as an electrode.

The content of MXene (MXene volume ratio) in the mixture was changed as shown in Tables 1 and 2, and the shape of the rubber base material in the metal mold was set to a brush type or a flat type to obtain electrodes of Examples 1 to 12 in Tables 1 and 2. In each of Comparative Example 1 in Table 1 and Comparative Example 2 in Table 2, an electrode was formed in the same manner as in Examples except that the MXene was not used.

2-2. Preparation of Electrode in which MXene is Partially Dispersed

First, a chip of an elastomer material EPDM and a carbon black powder were weighed so that carbon was 35 to 45 wt % (When the specific gravity of carbon black is 1.8 g/cm³ and the specific gravity of the elastomer material is 1 g/cm³, the volume ratio is about 23 to 31 vol %.) in the structure after molding, and were mixed in a dry manner to obtain a mixture. The mixture was charged into an injection molding machine, and an elastomer material in a state in which powder of carbon black was dispersed was injected into a metal mold in which a shape of a rubber base material was carved. The injection was followed by extrusion from the metal mold to obtain a conductive rubber base material for forming a MXene dispersed part.

Next, in a heated metal mold different from that at the time of producing the conductive rubber base material, a mixture of a chip of an elastomer material and powder obtained by dry-dispersing the MXene powder obtained in the above (6) was charged, and after they were melted, the conductive rubber base material was dipped for 5 minutes from above and pulled up and dried to obtain an electrode. The volume ratio of MXene in the partially dispersed part was 50 vol %.

By varying the time of dipping between 5 and 30 minutes, the MXene thickness was varied as shown in Tables 3 and 4, and the shape of the rubber base material in the metal mold was set to a brush type or a flat type to obtain electrodes of Examples 13 to 22 in Tables 3 and 4. Electrodes of Comparative Examples 1 and 2 in Tables 3 and 4 were prepared in the same manner as the electrodes of Comparative Examples 1 and 2 in Tables 1 and 2.

### 3. Measurement of Impedance

Using the electrodes of Examples 1 to 22, Comparative Example 1, and Comparative Example 2 described above, impedance was measured as the evaluation of conductivity as follows.

On a biosheet (product number: OHMTS01212) manufactured by WetLab, gel electrodes (product number: 019-415200) manufactured by Natus were installed as a counter electrode and a reference electrode. In addition, the electrode prepared in the above “2-1. Production of electrode in which MXene is entirely dispersed” or “2-2. Production of electrode in which MXene is partially dispersed” was installed as a working electrode.

Impedance measurement was performed using an electrochemical measuring instrument (product number: PGSTAT302N) manufactured by Metrohm. Detailed conditions are shown below. Basically, a signal around 10 Hz is important for brain waves, but a signal of 0.1 Hz may also be used. For example, the signal of slow potential, which is a hint for diagnosis of epilepsy, is 0.1 Hz. Therefore, in the measurement, values of 0.1 Hz and 10 Hz were used as representative values for analysis.

### (Impedance Measurement Conditions)

Frequency domain: 0.1 to 104 Hz

Number of plots: 51 points

Voltage: 10 mVrms

These measurement results are shown in Table 1 for the entire dispersion of MXene in which the electrode shape is a brush type, Table 2 for the MXene total dispersion in which the electrode shape is a flat type, Table 3 for the MXene partial dispersion in which the electrode shape is a brush type, and Table 4 for the MXene partial dispersion in which the electrode shape is a flat type.

TABLE 1

| | Electrode | | | | |
|---|---|---|---|---|---|
| | | | MXene volume ratio | Impedance [Ohm] | |
| | Shape | MXene dispersion form | (vol %) | 0.1 Hz | 10 Hz |
| Comparative | Brush type Soft Pulse | No MXene | 0 | 3100 | 2200 |
| Example 1 | | | | | |
| Example 1 | Brush type | Entirely dispersed | 10 | 4890 | 4200 |
| Example 2 | Brush type | Entirely dispersed | 20 | 2810 | 2650 |
| Example 3 | Brush type | Entirely dispersed | 40 | 1540 | 1340 |
| Example 4 | Brush type | Entirely dispersed | 60 | 1140 | 920 |
| Example 5 | Brush type | Entirely dispersed | 80 | 940 | 710 |
| Example 6 | Brush type | Entirely dispersed | 90 | 670 | 460 |

TABLE 2

| | Electrode | | | | |
|---|---|---|---|---|---|
| | | | MXene volume ratio | Impedance [Ohm] | |
| | Shape | MXene dispersion form | (vol %) | 0.1 Hz | 10 Hz |
| Comparative | Flat type Soft Pulse | No MXene | 0 | 3020 | 1980 |
| Example 2 | | | | | |
| Example 7 | Flat type | Entirely dispersed | 10 | 4600 | 4130 |
| Example 8 | Flat type | Entirely dispersed | 20 | 2790 | 2430 |
| Example 9 | Flat type | Entirely dispersed | 40 | 1530 | 1430 |
| Example 10 | Flat type | Entirely dispersed | 60 | 1030 | 880 |
| Example 11 | Flat type | Entirely dispersed | 80 | 930 | 700 |
| Example 12 | Flat type | Entirely dispersed | 90 | 660 | 440 |

TABLE 3

| | Electrode | | | | |
|---|---|---|---|---|---|
| | | | MXene thickness | | |
| | | | from skin | | |
| | | | contact surface | Impedance [Ohm] | |
| | Shape | MXene dispersion form | [μm] | 0.1 Hz | 10 Hz |
| Comparative | Brush type Soft Pulse | No MXene | 0 | 3100 | 2200 |
| Example 1 | | | | | |
| Example 13 | Brush type | Partially dispersed | 0.5 | 4300 | 2510 |
| Example 14 | Brush type | Partially dispersed | 1 | 1210 | 980 |
| Example 15 | Brush type | Partially dispersed | 12 | 1240 | 990 |
| Example 16 | Brush type | Partially dispersed | 93 | 1190 | 970 |
| Example 17 | Brush type | Partially dispersed | 1965 | 1180 | 960 |

TABLE 4

| | Electrode | | | | |
|---|---|---|---|---|---|
| | | | MXene thickness | | |
| | | | from skin | | |
| | | | contact surface | Impedance [Ohm] | |
| | Shape | MXene dispersion form | [μm] | 0.1 Hz | 10 Hz |
| Comparative | Flat type Soft Pulse | No MXene | 0 | 3020 | 1980 |
| Example 2 | | | | | |
| Example 18 | Flat type | Partially dispersed | 0.5 | 4230 | 2430 |
| Example 19 | Flat type | Partially dispersed | 1 | 1190 | 940 |
| Example 20 | Flat type | Partially dispersed | 14 | 1100 | 920 |
| Example 21 | Flat type | Partially dispersed | 89 | 1090 | 930 |
| Example 22 | Flat type | Partially dispersed | 1974 | 1140 | 930 |

The following can be seen from Tables 1 and 2. In both the cases where the shape of the electrode was the brush type and the flat type, when a very small amount of the layered material particle (MXene particle) was dispersed throughout the entire electrode, the impedance was rather higher than in the case without the layered material particle (MXene particle). However, in the case of entire dispersion, by containing 20 vol % or more of the layered material particle in the electrode, the impedance was sufficiently smaller than those in Comparative Examples 1 and 2.

In Example 6 and Example 12, the impedance was sufficiently smaller than those in Comparative Examples 1 and 2, respectively, but the mechanical strength was apparently insufficient for use as an electrode. From this, in consideration of securing the mechanical strength of the electrode, it can be said that the content is preferably 80 vol % or less when the layered material particle is dispersed in the entire electrode.

Tables 3 and 4 show the following. In both the cases where the shape of the electrode was the brush type and the flat type, when the MXene thickness was as small as less than 1 m, the impedance was rather higher than the case without the layered material particle (MXene particle). However, in the case of partial dispersion, when the MXene thickness was 1 μm or more, the impedance was sufficiently smaller than that in Comparative Examples 1 and 2.

The electrode of the present disclosure can be utilized for any suitable application, and can be preferably used as, for example, a biological signal sensing electrode.

### REFERENCE SIGNS LIST

1a, 1b Layer body (MₘXₙ layer)
3a, 5a, 3b, 5b Modification or termination T
7a, 7b MXene layer
10a, 10b MXene particle (layered material particle)
20a, 20b, 20c, 30, 40a, 40b, 40c, 40d, 50a, 50b Electrode
21, 31, 43 Base material part (may include support)
23 Silver/silver chloride electrode part
25 Conductive film
27 Tip region of contact pin
31,45 Support
33,47 Contact pin
41 Region formed of conductive composite material

1. An electrode in which at least a contact surface with an object to be measured is formed of a conductive composite material comprising:
a layered material particle comprising one or more layers, and an elastomer material, wherein
the one or more layers comprising a layer body represented by:
MₘXₙ
wherein M is one or more metals selected from Group 3, 4, 5, 6, or 7 and includes Ti;
X is a carbon atom, a nitrogen atom, or a combination thereof,
n is not less than 1 and not more than 4; and
m is greater than n but not more than 5,
and wherein a modification or termination T is present on a surface of the layer body,
wherein T is at least one selected from the group consisting of a hydroxyl group, a fluorine atom, a chlorine atom, an oxygen atom, and a hydrogen atom, and
wherein a volume ratio of the layered material particle in the conductive composite material is not less than 20 vol % and not more than 80 vol %.

2. The electrode according to claim 1, wherein the layered material particle is a metal atom-containing layered material particle containing a metal atom.

3. The electrode according to claim 2, wherein the metal atom is one or more of lithium, sodium, and potassium.

4. The electrode according to claim 1, wherein the electrode consists of the conductive composite material.

5. The electrode according to claim 1, further comprising a base material part containing carbon and the elastomer material and supporting the conductive composite material.

6. The electrode according to claim 1, wherein the conductive composite material has a thickness of 1 μm or more in a depth direction from the contact surface with the object to be measured.

7. An electrode having a contact surface with an object to be measured, the electrode comprising:
a conductive composite material forming at least the contact surface, the conductive composite material comprising:
a layered material particle comprising one or more layers, and an elastomer material,
the one or more layers comprising a layer body represented by:
MₘXₙ
wherein M is one or more metals selected from Group 3, 4, 5, 6, or 7 and includes Ti;
X is a carbon atom, a nitrogen atom, or a combination thereof,
n is not less than 1 and not more than 4; and
m is greater than n but not more than 5,
and a modification or termination T is present on a surface of the layer body,
wherein T is at least one selected from the group consisting of a hydroxyl group, a fluorine atom, a chlorine atom, an oxygen atom, and a hydrogen atom; and
a base material part supporting the conductive composite material and containing carbon and the elastomer material, wherein a volume ratio of the carbon in the base material part is not less than 20 vol % and not more than 60 vol %, and
a volume ratio of the layered material particle in the conductive composite material is not less than 20 vol % and not more than 80 vol %.

8. The electrode according to claim 7, wherein the layered material particle is a metal atom-containing layered material particle containing a metal atom.

9. The electrode according to claim 8, wherein the metal atom is one or more of lithium, sodium, and potassium.

10. The electrode according to claim 7, wherein the conductive composite material has a thickness of 1 μm or more in a depth direction from the contact surface with the object to be measured.

11. An electrode having a contact surface with an object to be measured, the electrode comprising:
a conductive composite material forming at least the contact surface, the conductive composite material comprising:
a layered material particle comprising one or more layers, and an elastomer material,
the one or more layers comprising a layer body represented by:
MₘXₙ
wherein M is one or more metals selected from Group 3, 4, 5, 6, or 7 and includes Ti;
X is a carbon atom, a nitrogen atom, or a combination thereof,
n is not less than 1 and not more than 4; and
m is greater than n but not more than 5,
and a modification or termination T is present on a surface of the layer body,
wherein T is at least one selected from the group consisting of a hydroxyl group, a fluorine atom, a chlorine atom, an oxygen atom, and a hydrogen atom; and
a base material part containing carbon and the elastomer material, wherein a volume ratio of the carbon in the base material part is not less than 20 vol % and not more than 60 vol %, and
the conductive composite material has a thickness of 1 μm or more in a depth direction from the contact surface with the object to be measured.

12. The electrode according to claim 11, wherein the layered material particle is a metal atom-containing layered material particle containing a metal atom.

13. The electrode according to claim 12, wherein the metal atom is one or more of lithium, sodium, and potassium.

14. The electrode according to claim 1, wherein the electrode is a brush-shaped electrode having a support and a plurality of contact pins configured to be brought into contact with the object to be measured, and the contact surface with an object to be measured is at least a part of a surface of at least one contact pin among the plurality of contact pins.

15. The electrode according to claim 1, wherein the contact surface with the object to be measured is porous.

16. The electrode according to claim 7, wherein the contact surface with the object to be measured is porous.

17. The electrode according to claim 11, wherein the contact surface with the object to be measured is porous.

18. The electrode according to claim 1, wherein the electrode has a disk shape, and the contact surface with the object to be measured is at least a part of a circular surface of the disk shape.

19. The electrode according to claim 1, further including a region formed of a silver/silver chloride electrode part.

20. The electrode according to claim 1, wherein the electrode is configured as a biological signal sensing electrode.
